(19) United States
(12) Patent Application Publication
(10) Pub. No.: US 2012/0251397 A1
(43) Pub. Date: Oct. 4, 2012

(54) ODOR CONTROL SYSTEM AND METHOD

(76) Inventor: Juergen T. Nick, Carefree, AZ (US)

(21) Appl. No.: 13/439,674

(22) Filed: Apr. 4, 2012

Related U.S. Application Data
(60) Provisional application No. 61/471,566, filed on Apr. 4, 2011.

(51) Int. Cl. A61L 9/00; B23P 17/04

(52) U.S. Cl. 422/120; 29/401.1

(57) ABSTRACT

The present invention relates to odor control systems and more particularly to a system and method for treating air to remove odors and return the treated air to the atmosphere. In one embodiment, an odor control system includes an ionization assembly, which has several ionizers producing an ionized air flow. The system also includes a foul air intake providing a foul air flow, such as from a wastewater treatment or other processing plant. The system also includes a reaction chamber, which provides a reaction volume for reaction of the foul air flow and the ionized air flow. The ionized air reacts with constituents in the foul air to treat the foul air for release of treated air to the environment.

### CROSS-REFERENCE TO RELATED APPLICATION(S)

This application claims priority to and the benefit of U.S. Provisional Application No. 61/471,566, filed Apr. 4, 2011, the entire contents of which are incorporated herein by reference.

### FIELD OF THE INVENTION

The present invention relates to odor control systems and more particularly to a system and method for treating air to remove odors and return the treated air to the atmosphere.

### BACKGROUND

In today's odor control market several different odor control systems are available. These systems commonly include, among others, chemical scrubbers, carbon scrubbers, and biological scrubbers, which utilize chemicals, carbon, or bacteria growing on an engineered or natural media, respectively. These systems typically include a vessel where the foul air contacts the carbon, chemical, or bacteria, and thus where the treatment of the air takes place. In a carbon system, the vessel includes the carbon media, such as carbon canisters. In a chemical system, the vessel contains the airstream into which the chemicals are sprayed. In a biological system, the vessel contains the biological growth media. The fouled air reacts with the carbon, chemical, or bacteria in this vessel, thereby treating the air and breaking down the odor-causing constituents. The treated air is then released to the atmosphere. A common constituent treated by this process is hydrogen sulfide (H₂S).

An example of a carbon odor removal system is the Phoenix® H₂S removal system provided by Calgon Carbon Corporation (Pittsburgh, Pa.). An example of a chemical odor control system is the LO/PRO® Packaged Odor Control System provided by Siemens Water Technologies (San Diego, Calif.). An example of a biological scrubber is the MEGTEC® Bioscrubber provided by MEGTEC Systems, Inc.

Existing chemical scrubbers often have a high cost associated with the ongoing purchase of chemicals as well as high maintenance and replacement costs for chemical feed pumps and instruments.

Carbon scrubbers have relatively high power consumption due to the head loss created by the carbon bed through which the foul air passes and in which the constituents such as H₂S are absorbed. In addition, carbon has a limited life expectancy which varies as a function of constituent loading and other factors. A higher H₂S loading leads to a shorter carbon life span. Thus the carbon canisters are replaced regularly and at a relatively high cost. In addition, disposal costs can be high, as spent carbon often must be treated as hazardous waste.

Biological scrubbers may occupy a larger space per air flow volume than carbon or chemical scrubbers. Additionally, ongoing costs are incurred as the biological carrier media is replaced approximately every 10 years in the case of an engineered media and more frequently in the case of a natural media such as wood chips or shells.

Accordingly, a need exists for an improved odor control system to treat and clean foul air without relying on consumable carbon, chemical, and biological media.

### SUMMARY

The present invention relates to odor control systems and more particularly to a system and method for treating air to remove odors and return the treated air to the atmosphere. According to an embodiment of the invention, foul air is treated by mixing with ionized air to break down the odor-causing constituents, such as H₂S (converted into sulfur and water) or ammonia. These constituents may also be referred to as volatile organic compounds (VOC). The system includes an air flow combiner that combines the foul air with the ionized air, and a reaction volume where the mixed air reacts. The ionized air reacts with the constituents in the foul air, converting them into innocuous components. The treated air can then be released to the atmosphere. The odor control system with ionizers produces no waste byproducts such as spent carbon, chemicals, or biological media, and does not require replacement of such consumables. The system operates to treat the foul air with only the continued maintenance and operation of the ionizers.

In one embodiment, an odor control system includes an ionization assembly, which has several ionizers producing an ionized air flow. The system also includes a foul air intake providing a foul air flow, such as from a wastewater treatment or other processing plant. The system also includes a reaction chamber, which provides a reaction volume for reaction of the foul air flow and the ionized air flow. The ionized air reacts with constituents in the foul air to treat the foul air for release of treated air to the environment.

In one embodiment, a method for converting an existing odor control system into an ionization-based odor control system includes providing an existing chemical, carbon, or biological odor control system having a reaction vessel and a reaction agent, and then discarding the reaction agent. The method includes installing an ionization assembly, and coupling the ionization assembly to the reaction vessel. The ionization assembly includes one or more ionizers and an ionized air volume.

In one embodiment, a method for removing odor-causing constituents includes providing an odor control system, which includes an ionization assembly, a foul air intake, and a reaction chamber. The ionization assembly has a plurality of ionizers. The method also includes passing a first foul air flow into the foul air intake and into the reaction chamber. The foul air has odor-causing constituents that need to be treated. The method includes ionizing a second air flow to produce an ionized air flow, and passing the ionized air flow into the reaction chamber. Then, the method includes reacting the foul air and the ionized air in the reaction chamber to treat the odor-causing constituents.

### BRIEF DESCRIPTION OF THE DRAWINGS

FIG. 1 is a perspective view of an odor control system according to an embodiment of the invention.

FIG. 2 is a top view of the system of FIG. 1.

FIG. 3 is a cut-away top view of the system of FIG. 1.

FIG. 4 is a cut-away side elevational view of the system of FIG. 1.

FIG. 5A is a side view of an odor control system according to an embodiment of the invention.

FIG. 5B is a top view of the system of FIG. 5A.

FIG. 5C is a side view of an odor control system according to an embodiment of the invention.

FIG. 5D is a top view of the system of FIG. 5C.

FIG. 6 is a schematic view of airflow through an odor control system according to an embodiment of the invention.

### DETAILED DESCRIPTION

The present invention relates to odor control systems and more particularly to a system and method for treating air to remove odors and return the treated air to the atmosphere. According to an embodiment of the invention, foul air is treated by mixing with ionized air to break down the odor-causing constituents, such as H₂S (converted into sulfur and water) or ammonia. These constituents may also be referred to as volatile organic compounds (VOC). The system includes an air flow combiner that combines the foul air with the ionized air, and a reaction volume where the mixed air reacts. The ionized air reacts with the constituents in the foul air, converting them into innocuous components. The treated air can then be released to the atmosphere. The odor control system with ionizers produces no waste byproducts such as spent carbon, chemicals, or biological media, and does not require replacement of such consumables. The system operates to treat the foul air with only the continued maintenance and operation of the ionizers.

In a further embodiment, an existing odor control system, such as a chemical, carbon, or biological system, can be modified to be converted into an ionization-based system. The existing vessel where the foul air is treated by contact with sprayed chemicals, carbon canisters, or biological media is converted into a reaction chamber for contact between the ionized air and foul air, and an ionization assembly is installed, as described in further detail below. Accordingly, an existing odor control system can be converted into an efficient ionization-based system. This conversion can reduce the energy consumption of the system as well as provide other environmental benefits.

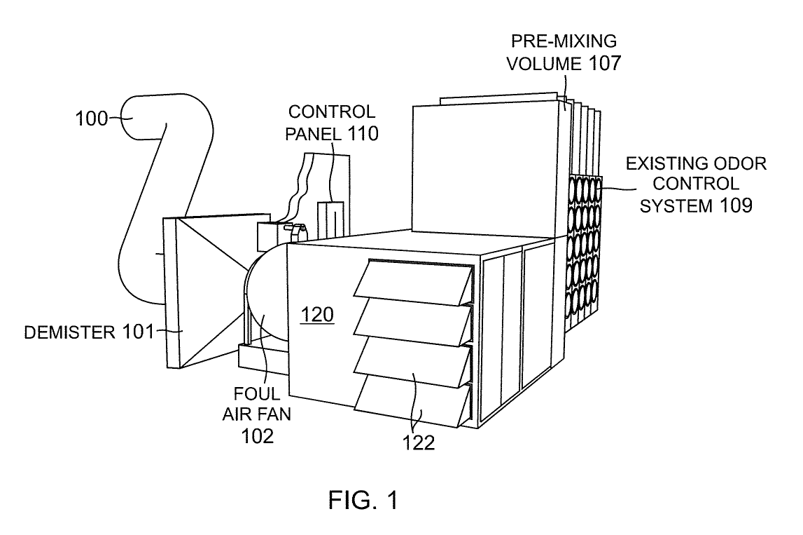

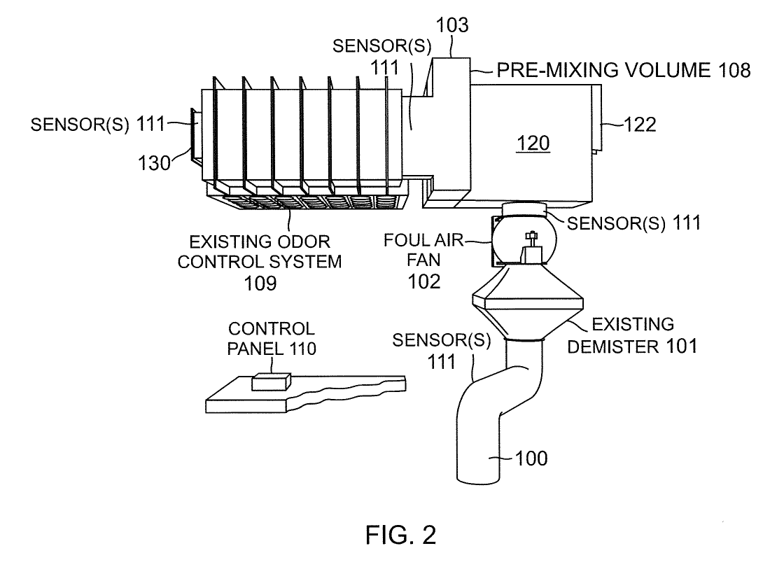

An embodiment of the invention is shown in FIGS. 1-4. The system includes a conduit or duct 100 leading from the source of foul air, referred to as the process area, which may be a treatment plant, pump station, storage tank, industrial housing, or other system. Examples include tanks and equipment used in wastewater treatment plants and other industrial processes. The foul air is moved by pumps or fans through this conduit 100 and through an optional demister 101. The demister 101 removes moisture from the foul air prior to treatment. The demister may be part of an existing odor control system, or it may be installed to the converted system if desired. One or more sensors 111 such as moisture sensors or hydrogen sulfide H₂S sensors may be placed in the air flow path upstream of the demister, as shown in FIG. 2, or at any other suitable location in the system.

The system includes a foul air fan 102 downstream of the demister 101 and conduit 100. If the system is a conversion of an existing system, the prior existing fan may be used for this purpose, or a new foil air fan 102 may be installed. The foul air fan 102 includes a motor and a fan sized appropriately to convey the foul air from the process area into the air treatment system. The fan and motor are sized according to the air flow volume of the particular process area.

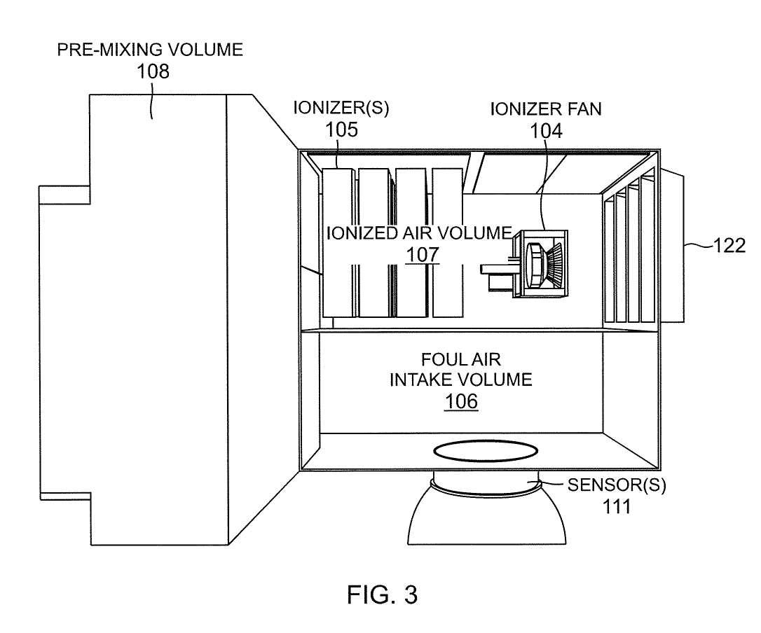

The fan 102 conveys air from the process area to a foul air intake volume 106 (see FIG. 3). From there the foul air is directed into an air flow combiner 103, described in further detail below.

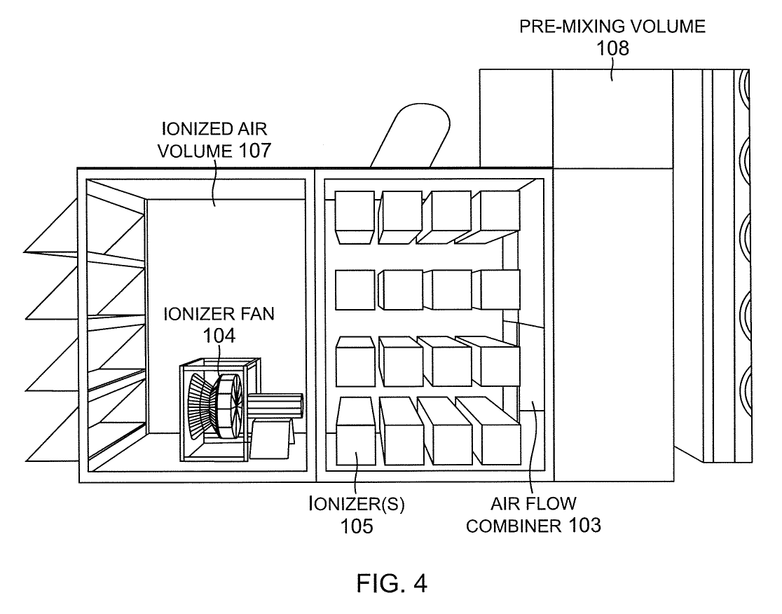

The ionized air used for the air treatment is produced by an ionizer assembly, which includes an ionizer fan 104, one or more ionizers 105, and an ionized air volume 107 (see FIGS. 3-4). The ionized air volume 107 includes an inlet such as vents 122 at one end, where air is drawn from the atmosphere by the fan 104. This air is passed over the ionizers 105, which apply high voltage to ionize the air molecules. The ionizers 105 generate ions and potentially small amounts of ozone. The ionizers 105 may be any suitable and commercially available ionization system. The ionizer fan 104 conveys the ions (and possibly a small amount of ozone) from the ionized air volume 107 into the air flow combiner 103.

The volume inside the air flow combiner 103 may be referred to as the pre-mixing volume 108, where the ions/ozone and foul air first meet. The air flow combiner 103 brings the two airflows together and directs the mixing airflow toward a reaction chamber or vessel 109, where the ions react with the constituents in the foul air. The air flow combiner 103 may be, for example, a duct or conduit that connects the two volumes 106, 107 and leads to the reaction chamber 109.

The intake volume 106, ionized air volume 107, and the pre-mixing volume 108 may be included within a combined housing 120, as shown in FIGS. 1-2. Alternatively, these components may be separate, and connected together by conduits to direct the respective air flows.

In the reaction chamber 109, the ions (and possibly ozone) react with the VOC or odor-causing constituents. For example, the negative ions attract and break down H₂S into sulfur and water. In the chamber 109, the mixture of foul air and ionized air reacts for a certain amount of time before the air exits the chamber 109 at outlet 130 to the atmosphere. The reaction chamber 109 is sized to provide the desired reaction time, according to the requirements of the process area and the rate and volume of air flow. In one embodiment, the reaction chamber 109 has a volume of about 1,000 cubic feet, and the air flow rate provides a time within the chamber of about one minute. In another embodiment, the reaction time is around 5-10 seconds, and in another embodiment within a range of about 2 seconds to one minute. The output of the ionizers 105 can also be adjusted based on the available contact time in order to provide sufficient treatment of the foul air. Additionally, the volume of the vessel 109 can be increased to increase the reaction time.

The odor control system is further equipped with a controller including a control panel 110 (see FIGS. 1-2) to control the operation and functions of the system. The control panel 110 contains components such as (but not limited or restricted to) a programmable logic controller (PLC), variable frequency (VFD) or adjustable frequency (AFD) drives, a touch screen, push buttons, measuring instruments, analyzers, terminal blocks, contactors, and other instruments or tools. The control panel is capable of sending, receiving, processing, and recording signals from the drives, fans, sensors, instruments, ionizers, and other components.

Sensors 111 may be provided at various locations within the system, for example, upstream of the demister, upstream of the foul air intake volume, within the pre-mixing volume 108, at the system outlet 130, or any other location as deemed necessary (see FIG. 2). These sensors can be used to measure air quality parameters between process steps. For example, these sensors can measure H₂S content, ozone (O₃) levels, and ion count. These parameters can be input into the controller, which then uses these measurements to control the speeds of the foul air fan 102 or the ionizer fan 104, to increase or decrease power to the ionizers, including switching ionizers on and off, to increase or decrease the quantity or level of ionization, or to collect measurements for reporting, compliance, maintenance records, efficiency analysis, and other data acquisition purposes.

Notably, the ionizers 105 are located upstream of the reaction chamber 109 and upstream of the first mixing volume 108 where the foul air is added to the ionized air flow. This configuration is provided in order to prevent exposure of the ionizers to the foul air, which could lead to decomposition or corrosion of the ionizers. Dry, ambient air is drawn into the ionizer volume from the atmosphere, optionally through a filter. Downstream of the ionizers, the ionized air mixes with the foul air and moves into the reaction chamber, where there is no particular moisture requirement, and no working equipment such as the ionizers. The reaction chamber does not include any working parts or consumable media that needs to be frequently maintained or replaced. Thus the reaction between the foul and ionized air that takes place in this chamber is efficient and sustainable, with the ionized air input.

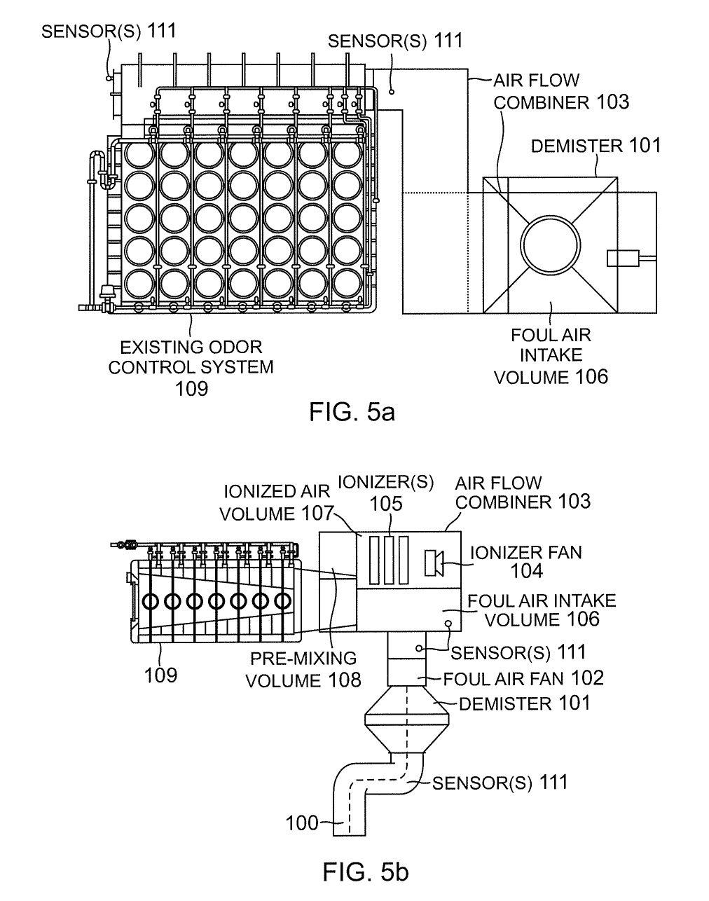

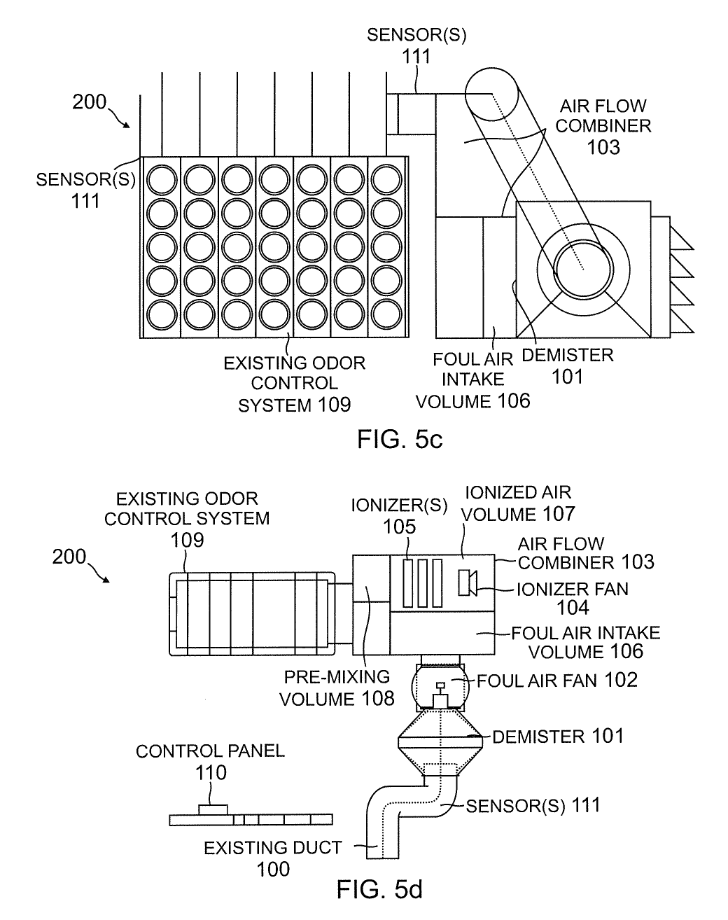

FIGS. 5A and 5B show another embodiment of an odor control system, including a reaction vessel or volume from an existing odor control system 109 with an added ionization assembly including ionizers 105, ionized air volume 107, and ionizer fan 104. The ionization assembly is included with a foul air intake volume 106 and pre-mixing volume 108 within a unified housing referred to collectively as the air flow combiner 103.

When the odor control system is a retro-fit or conversion of an existing system, the reaction chamber or vessel 109 may be part of an existing odor control system, such as, for example, the reaction volume that previously housed the carbon canisters, biological growth medium, or chemically-sprayed air. The existing odor control system may be retro-fitted and converted into an ionization system by utilizing this vessel 109 and providing an ionization assembly. The foul air fan 102 may not be required to be replaced. Further, the foul air intake volume 106 and premixing volume 108 may be part of the existing odor control vessel 109 and therefore air flow combiner 103 may consist only of the ionized air volume 107. Other variations and configurations of these volumes and vessels are possible, depending on the configuration of the existing system.

FIGS. 5C and 5D show an odor control system 200 according to another embodiment of the invention, with an existing odor control system 109 retro-fitted with an ionization assembly including ionizers 105.

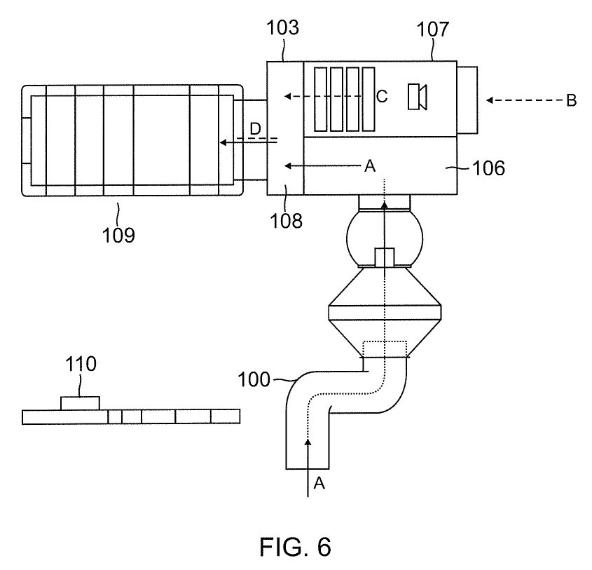

FIG. 6 shows a schematic view of the airflow through an odor control system according to another embodiment. Foul air is indicated by the solid arrows A, and is drawn from the conduit 100 into the intake volume 106 and into the pre-mixing volume 108. Atmospheric air indicated by dashed line B is drawn into the ionization volume 107, where it is ionized. Ionized air indicated by the dashed line C flows from there into the pre-mixing volume 108. The mixed foul and ionized air then flows into the reaction chamber 109, as indicated by the mixed arrow D.

In one embodiment, a method of converting an existing odor control system into an ionization-based odor control system is provided. An existing chemical, carbon, biological, or other system can be converted into an ionization-based system. The method includes removing and discarding the spent carbon, the biological media, or the chemical sprayers. The reaction vessel where the foul air previously reacted with these agents is then sealed and connected to the air flow combiner 103. An ionization assembly is provided and connected upstream of the air flow combiner. The conduit 101 from the process area is directed to the air flow combiner via an air intake volume and ducts or conduits as necessary. A demister may be included in this air flow if desired. Additionally, a foul air fan may be added to direct the foul air through this system to the air flow combiner 103. Sensors may be provided at various desired locations along each air flow path, and may be connected to a controller. The controller can then be used to measure and monitor air flow parameters and control the speed of the fans and the output of the ionizers. A control panel may also be installed to provide access and input to the controller, as well as outputs or displays showing the status of various components in the system, and measurements from the sensors.

With the conversion of the reaction vessel, disposal of the prior reactive agents, and installation of the ionization assembly, the prior existing odor treatment system can be converted into the ionization-based system described here. Systems that previously relied on chemical sprayers, carbon canisters, and biological growth media can be retrofitted into this ionization system. The conversion method is flexible to accommodate the various configurations and components of the existing system, adding ducts, fans, and additional volumes as needed, so that existing components can be re-used and re-routed into the new system. The conversion utilizes components of the prior existing system to make the conversion more efficient and cost-effective.

Whether a new installation or a conversion of an existing system, the odor control system effectively and efficiently treats the foul air to break down the odor-causing constituents and return the treated air to the atmosphere. The system accomplishes this treatment without consuming carbon, chemicals, or biological growth media. The system can continue to treat the foul air as long as the ionizers are maintained and operated. The odor control system reduces waste created by the air treatment and reduces the costs to operate the system. The system can also utilize existing components, such as reaction vessel 109, and utilizes this volume to provide sufficient contact time to treat the foul air.

In addition, the ionization-based odor control system accomplishes the desired air treatment with less impact on the environment as compared to many prior systems. For example, the ionization-based system consumes less energy than corresponding carbon scrubbers, as the ionizers consume less power. The conversion to an ionization-based system, as described above, can reduce the energy consumption of the system. Additionally, the reaction between the ionized air and the foul air breaks down the odor-causing constituents or VOC rather than simply capturing and discarding these constituents. The ionizers accomplish this result without the use of hazardous chemicals and without creating hazardous waste products such as spent carbon or other biological agents. Thus, prior carbon, chemical, biological, or other systems can be converted into an ionization-based system, as provided herein, in order to reduce energy consumption and reduce the environmental impact of the system.

Although the present invention has been described and illustrated in respect to exemplary embodiments, it is to be understood that it is not to be so limited, since changes and modifications may be made therein which are within the full intended scope of this invention as hereinafter claimed.

What is claimed is:

1. An odor control system comprising:
an ionization assembly including one or more ionizers producing an ionized air flow;
a foul air intake providing a foul air flow; and
a reaction chamber providing a reaction volume for reaction of the foul air flow and the ionized air flow, wherein the ionized air reacts with constituents in the foul air to treat the foul air for release of treated air to the environment.

2. The odor control system of claim 1, wherein the ionization assembly further comprises an ionizer fan for drawing air toward the ionizers.

3. The odor control system of claim 1, wherein the ionization assembly and the foul air intake are provided in a combined housing.

4. The odor control system of claim 3, wherein the combined housing comprises a pre-mixing volume where the foul air flow meets the ionized air flow, and wherein the pre-mixing volume is upstream from the reaction chamber.

5. The odor control system of claim 1, further comprising a pre-mixing volume where the foul air flow meets the ionized air flow, and wherein the ionizers are located upstream of the pre-mixing volume.

6. The odor control system of claim 5, wherein the ionizers are isolated from the foul air flow.

7. The odor control system of claim 1, wherein the reaction chamber is devoid of consumable media for treating the foul air flow.

8. The odor control system of claim 1, wherein the ionization assembly comprises a housing having an ionized air volume, a foul air intake volume, and a pre-mixing volume, and wherein the foul air intake volume and the ionized air volume are fluidically isolated from each other and are fluidically connected to the pre-mixing volume.

9. The odor control system of claim 1, wherein the reaction chamber comprises a reaction vessel of a pre-existing chemical, carbon, or biological odor control system.

10. A method for converting an existing odor control system into an ionization-based odor control system, comprising:
providing an existing chemical, carbon, or biological odor control system having a reaction vessel and a reaction agent;
discarding the reaction agent;
installing an ionization assembly including one or more ionizers and an ionized air volume; and
coupling the ionization assembly to the reaction vessel.

11. The method of claim 10, wherein discarding the reaction agent comprises discarding one of a chemical spray, a carbon scrubber, or a biological media.

12. The method of claim 10, wherein installing an ionization assembly comprises providing a pre-mixing volume upstream of the reaction vessel, for mixing of a foul air flow and an ionized air flow.

13. The method of claim 10, wherein installing an ionization assembly comprises installing the ionizers upstream of the reaction vessel.

14. The method of claim 10, further comprising providing a controller for processing signals from the ionization assembly.

15. A method for removing odor-causing constituents, comprising:
providing an odor control system comprising
an ionization assembly including one or more ionizers;
a foul air intake; and
a reaction chamber providing a reaction volume;
passing a first foul air flow comprising odor-causing constituents into the foul air intake and into the reaction chamber;
ionizing a second air flow to produce an ionized air flow;
passing the ionized air flow into the reaction chamber; and
reacting the foul air and the ionized air in the reaction volume to treat the odor-causing constituents.
